(19) United States
(12) Patent Application Publication
(10) Pub. No.: US 2013/0165994 A1
(43) Pub. Date: Jun. 27, 2013

(54) MAINTAINING STIMULATION THERAPY EFFICACY

(71) Applicants: David J. Ternes, Roseville, MN (US); Jason J. Hamann, Blaine, MN (US); Stephen Ruble, Lino Lakes, MN (US)

(72) Inventors: David J. Ternes, Roseville, MN (US); Jason J. Hamann, Blaine, MN (US); Stephen Ruble, Lino Lakes, MN (US)

(21) Appl. No.: 13/705,446

(22) Filed: Dec. 5, 2012

Related U.S. Application Data
(60) Provisional application No. 61/580,422, filed on Dec. 27, 2011.

(51) Int. Cl. A61N 1/36

(52) U.S. Cl. CPC A61N 1/36139
USPC 607/59; 607/60

(57) ABSTRACT

An apparatus comprises a therapy circuit configured to provide electrical neural stimulation therapy to a subject using a first set of a plurality of implantable electrodes, a switching circuit communicatively coupled to the therapy circuit and configured to change the delivery of therapy among the plurality of implantable electrodes, and a control circuit. The control circuit is configured to initiate delivery of the neural stimulation therapy to a first subset of the first set of electrodes during a therapy session, change the therapy delivery from the first subset of electrodes to at least a second subset of electrodes of the first set of the plurality of implantable electrodes, and recurrently alternate the therapy delivery between the first and second subsets of electrodes during the same therapy session.

### CLAIM OF PRIORITY

This application claims the benefit of priority under 35 U.S.C. §119(e) of Ternes et al., U.S. Provisional Patent Application Ser. No. 61/580,422, filed on Dec. 27, 2011, the benefit of priority of which is claimed hereby, and is incorporated by reference herein in its entirety.

### BACKGROUND

Neural stimulation, such as vagus nerve stimulation, has been proposed as a therapy for a number of conditions. Examples of neural stimulation therapies include neural stimulation therapies for respiratory problems such as sleep disordered breathing, blood pressure control such as to treat hypertension, cardiac rhythm management, myocardial infarction and ischemia, heart failure (HF), epilepsy, depression, pain, migraines, eating disorders and obesity, and movement disorders.

### OVERVIEW

This document relates generally to systems, devices, and methods that provide electrical neural stimulation therapy to a patient or subject. In particular it relates to systems, devices, and methods that maintain electrical therapy efficacy even when electrodes are non-optimally placed.

An apparatus example can include a therapy circuit configured to provide electrical neural stimulation therapy (NST) to a subject using a first set of a plurality of implantable electrodes, a switching circuit communicatively coupled to the therapy circuit and configured to change the delivery of therapy among the plurality of implantable electrodes, and a control circuit. The control circuit is configured to initiate delivery of the NST to a first subset of the first set of electrodes during a therapy session, change the therapy delivery from the first subset of electrodes to at least a second subset of electrodes of the first set of the plurality of implantable electrodes, and recurrently alternate the therapy delivery between the first and second subsets of electrodes during the same therapy session.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is an illustration of an example of portions of a system that uses an IMD.

FIG. 2 shows an illustration of an example of a multi-electrode lead for stimulating nerves of the spinal cord.

FIG. 3 shows an illustration of two other examples of multi-electrode leads.

FIG. 4 shows an illustration where a multi-electrode lead experiences a shift in position.

FIG. 5 shows a flow diagram of an example of a method of maintaining efficacy of autonomic neural modulation therapy.

FIG. 6 shows a block diagram of portions of an example of medical device that maintains efficacy of autonomic neural modulation therapy.

FIG. 7 shows a block diagram of portions of another example of a medical device that maintains efficacy of autonomic neural modulation therapy.

### DETAILED DESCRIPTION

This document discusses systems and methods for delivering electrical neural modulation therapy. A medical device can include one or more of the features, structures, methods, or combinations thereof described herein. For example, a neural stimulator may be implemented to include one or more of the advantageous features or processes described below. Such a device may be implemented to provide a variety of therapeutic or diagnostic functions.

Neural Stimulation Therapy (NST) can include autonomic modulation therapy (AMT). AMT involves the stimulation of the autonomic nervous system. For example, electrical stimulation of neural targets within the autonomic nervous system may be used to deliver AMT.

The autonomic nervous system (ANS) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscles around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system. Afferent nerves convey impulses toward a nerve center, and efferent nerves convey impulses away from a nerve center.

Stimulating the sympathetic and parasympathetic nervous systems can cause heart rate, blood pressure and other physiological responses. For example, stimulating the sympathetic nervous system dilates the pupil, reduces saliva and mucus production, relaxes the bronchial muscle, reduces the successive waves of involuntary contraction (peristalsis) of the stomach and the motility of the stomach, increases the conversion of glycogen to glucose by the liver, decreases urine secretion by the kidneys, and relaxes the wall and closes the sphincter of the bladder. Stimulating the parasympathetic nervous system (inhibiting the sympathetic nervous system) constricts the pupil, increases saliva and mucus production, contracts the bronchial muscle, increases secretions and motility in the stomach and large intestine, and increases digestion in the small intestine, increases urine secretion, and contracts the wall and relaxes the sphincter of the bladder. The functions associated with the sympathetic and parasympathetic nervous systems are many and can be complexly integrated with each other.

A reduction in parasympathetic nerve activity contributes to the development and progression of a variety of cardiovascular diseases. Some embodiments of the present subject matter can be used to prophylactically or therapeutically treat various cardiovascular diseases using AMT to stimulate nerves and thereby modulate autonomic tone. Neural stimulation to treat cardiovascular diseases can be referred to as neurocardiac therapy (NCT). Vagal stimulation used to treat cardiovascular diseases may be termed either vagal stimulation therapy (VST) or NCT. However, VST may be delivered for non-cardiovascular diseases, and NCT may be delivered by stimulating a nerve other than the vagal nerve. Both VST and NCT are examples of AMT.

NCT, by way of example and not limitation, includes the stimulation of an autonomic neural target to provide a therapy for a cardiac arrhythmia, ischemia, heart failure, angina, atherosclerosis, blood pressure, and the like. By way of example and not limitation, autonomic neural targets used to deliver NCT include the vagus nerve, cardiac branches of the vagal nerves, the carotid sinus nerve, baroreceptors such as baroreceptors in the carotid sinus or baroreceptors in the pulmonary artery, chemoreceptors, cardiac fat pads, the spinal column or some nerve roots extending from the spinal column. Examples of cardiovascular diseases or conditions include hypertension, HF, and cardiac remodeling. These conditions are briefly described below.

Hypertension is a cause of heart disease and other related cardiac co-morbidities. Hypertension occurs when blood vessels constrict. As a result, the heart works harder to maintain flow at a higher blood pressure, which can contribute to HF. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease. A large segment of the general population, as well as a large segment of patients implanted with pacemakers or defibrillators suffer from hypertension. The long term mortality as well as the quality of life can be improved for this population if blood pressure and hypertension can be reduced. Many patients who suffer from hypertension do not respond to treatment, such as treatments related to lifestyle changes and hypertension drugs.

HF refers to a clinical syndrome in which cardiac function causes a below normal cardiac output that can fall below a level adequate to meet the metabolic demand of peripheral tissues. HF may present itself as congestive heart failure (CHF) due to the accompanying venous and pulmonary congestion. HF can be due to a variety of etiologies such as ischemic heart disease. HF patients have impaired autonomic balance, which is associated with LV dysfunction and increased mortality.

Cardiac remodeling refers to a complex remodeling process of the ventricles that involves structural, biochemical, neurohormonal, and electrophysiologic factors, which can result following a myocardial infarction (MI) or other cause of decreased cardiac output. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle may ensue that causes further and progressive dilatation. As the heart begins to dilate, afferent baroreceptor and cardiopulmonary receptor signals are sent to the vasomotor central nervous system control center, which responds with hormonal secretion and sympathetic discharge. The combination of hemodynamic, sympathetic nervous system and hormonal alterations (such as presence or absence of angiotensin converting enzyme (ACE) activity) account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction. It has been shown that the extent of ventricular remodeling is positively correlated with increased mortality in post-MI and heart failure patients.

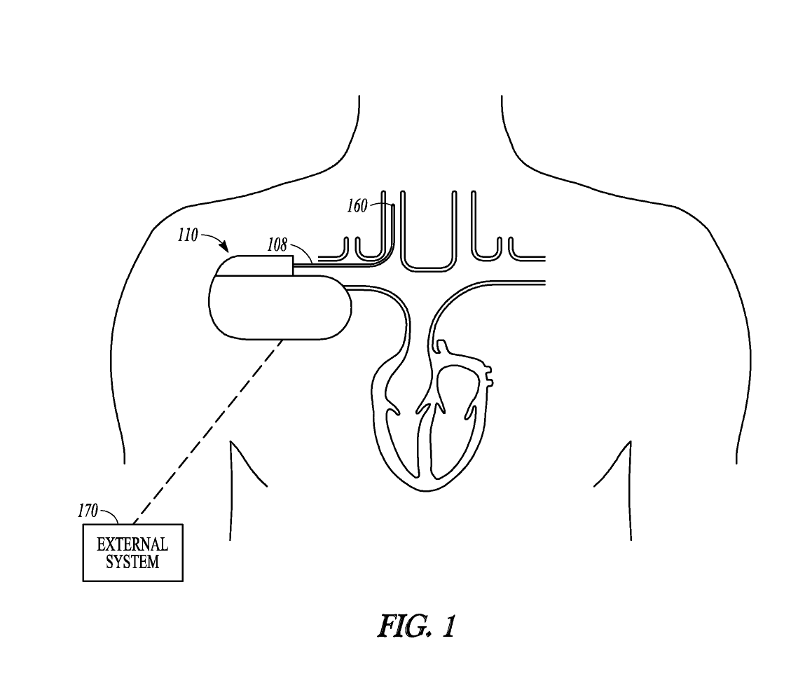

FIG. 1 is an illustration of an example of portions of a system that uses an IMD 110 to provide NST (e.g., AMT). The IMD 110 may include one or more leads 108 to provide the NST. The neural stimulation lead 108 is designed for placement to provide therapy to specific areas of the nervous system and includes one or more electrodes 160. Electrodes of the lead can be positioned in blood vessel proximate to a nerve trunk or nerve bundle so that electrical stimulation passes through a vessel wall to stimulate the nerve. For instance, neural stimulation lead 108 may be positioned in the jugular vein. Such a placement of electrodes may be useful for stimulation of the vagus nerve.

Other placements involve positioning a neural stimulation lead near the carotid artery sheath. The carotid sheath is fibrous connective tissue that surrounds a vascular compartment in the neck containing the jugular vein and the carotid artery. These electrode configurations may be useful for placement proximal the vagus nerve. Descriptions of systems and methods to provide baroreflex stimulation can be found in U.S. Pat. No. 8,000,793, by Libbus et al., filed May 23, 2008, and entitled “Automatic Baroreflex Modulation Based on Cardiac Activity,” which is incorporated herein by reference in its entirety.

The IMD 110 may deliver intermittent neural stimulation. For example, intermittent neural stimulation may be delivered to treat chronic diseases such as heart failure and hypertension. Intermittent neural stimulation can be delivered using a duty cycle of a stimulation period. Each duty cycle can include a train of neural stimulation pulses. The duty cycle and stimulation period need not be constant throughout the Neural Stimulation Therapy (NST). For example, the duration or frequency of the duty cycle can be adjusted to adjust an intensity of the NST. Also, the start and/or stop of the duty cycle can be dependent on enabling conditions. The duty cycle and/or stimulation period can be adjusted in every subsequent stimulation period. Unless expressly disclosed otherwise herein, “stimulation period” and “duty cycle” are not intended to only encompass constant values that result in neural stimulation in a precise periodic manner, but rather is intended to include intermittent neural stimulation where therapeutically-effective or prophylactically-effective neural stimulation is delivered for a time and then not delivered for a time, and then delivered for a time. In electrical stimulation, for example, a train of neural stimulation pulses (current or voltage) can be delivered during a duty cycle of stimulation.

The system can also include an IMD programmer or other external system 170 that communicates wireless signals 190 with the IMD 110, such as by using radio frequency (RF) signals, inductive signals, or other telemetry signals. The external system 170 can include an external device that communicates with a remote system via a network, such as a computer network or cellular phone network. In some examples, the remote system provides patient management functions and may include one or more servers to perform the functions.

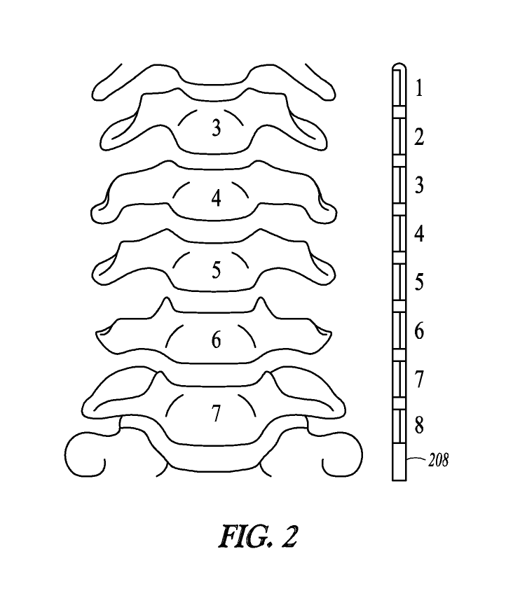

FIG. 2 shows an illustration of an example of a multi-electrode lead 208 for stimulating nerves of the spinal cord. Spinal cord stimulation (SCS) includes neural stimulation that is sometimes used in pain management. Note that the lead and electrodes are not drawn to the same scale as the spinal cord. The multi-electrode lead 208 may also be disposed in the azygos vein for stimulation of the nerves of the spinal cord. The multi-electrode lead 208 may also be used for vagus nerve stimulation and disposed for this purpose in a blood vessel located in the neck or near the carotid artery sheath.

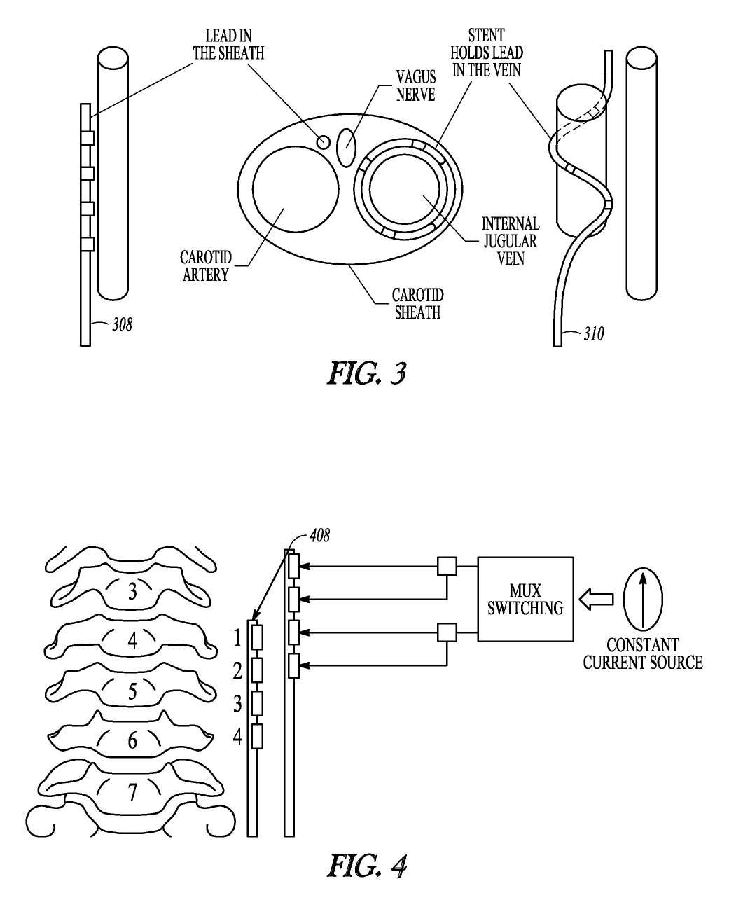

FIG. 3 shows an illustration of examples of multi-electrode leads 308 and 310. The illustration shows two examples of placing a lead in a vessel or other structure proximal a nerve or nerve site. Lead 308 is a multi-electrode lead configured by shape and size for placement in the carotids sheath of the subject. The carotid sheath is fibrous connective tissue that surrounds a vascular compartment in the neck containing the jugular vein and the carotid artery. Lead 308 is shown positioned proximal the vagus nerve in the cross section view shown the Figure. Lead 308 is sometimes referred to as an intra-sheath lead.

Lead 310 is a multi-electrode lead configured for placement in the jugular vein. The lead 310 can include a mechanical bias to place the electrodes at the edge of the jugular vein or, as shown in the Figure, can be positioned using a stent. Electrodes of the lead are shown positioned proximal to the vagus nerve in the cross section view such as to provide transvascular stimulation. Lead 310 is sometimes referred to as an intra-vascular lead. Note that although specific arrangements of leads are shown in FIGS. 1-3, the present methods and systems apply to a variety of leads, such as any type of neural stimulation lead or electrode that is targeting a nerve or nerve site and is placed proximal to the target location but is not affixed to, or around, the target location. Neural stimulation leads can also be subcutaneous, percutaneous, and transcutaneous leads. In some examples, neural stimulation leads can include one or more electrodes for implantation in a lymphatic vessel.

In various examples, at least one lead used for neural stimulation is wirelessly coupled to the neural stimulation circuitry such that the neural stimulation circuitry wirelessly applies electrical stimulation to the electrode. Such wireless embodiments provide additional flexibility in placement of the electrode(s) and stimulating device. In certain examples, at least one electrode is integrated or otherwise formed on the housing of the neural stimulating device such that the neural stimulation circuitry applies electrical stimulation via the electrode on the housing.

In various examples, neural stimulation is provided using multiple neural stimulation devices or units. The neural modulation units may be configured to communicate with each other (e.g., wirelessly) over a telemetry communication channel and with an external programmer device. Each unit can be configured for one or both of providing neural stimulation and sensing modalities for measuring a plurality of physiological variables. The neural modulation units may be disposed at different anatomical locations to enable different types of neural stimulation in accordance with shared variable measurements.

In some examples, neural stimulation therapy can include peripheral nerve field modulation therapy, or PNFM. In PNFM, a neural therapy delivery system and electrodes are configured to deliver current at one or more peripheral nerve fields, or are configured to control the field potentials at one or more peripheral fields, or are configured to both deliver current at one or more peripheral nerve fields and control the field potentials at one or more peripheral fields.

Neural stimulation leads may move due to the routine physical movement of the subject, such as lying down, sitting down, standing, and walking This movement of the leads may reduce efficacy of the stimulation therapy because electrodes of the lead may not be at the desired target electrode positions.

FIG. 4 shows an illustration where a multi-electrode lead 408 experiences a shift in position or location. In the illustration, NST is targeted to the area of vertebrate numbered 3-5 and therapy to those areas is intended to be provided by electrodes numbered 3-4. But because of the movement of the lead 408, electrodes 3-4 may end up positioned beyond the most effective range for providing the therapy. The example in the Figure illustrates a shift downward. The shift may also be outward from the target position, or involve a rotation away from the target location or area, such as when the lead is disposed in the neck and the neck is rotated. As a result of the shift, some electrodes may shift out of the target area and other electrodes may shift into the target area.

NST stimuli are often delivered at a desired frequency that is believed to be most effective. However, there can be a range of frequencies where the therapy is still 75-90 percent as effective as the desired frequency. For instance, 60 Hertz (Hz) may be the desired frequency for the NST therapy, but therapy in the range 10-50 Hz may be 75-90% as effective.

A solution to the shifting lead placement shown in FIG. 4 can be to multiplex the constant current stimuli of the NST between the pair of electrodes numbered 1-2 and 3-4. If the frequency of the stimulus pulses is 60 Hz and the pulses are evenly multiplexed so that half of the pulses are applied using electrodes 1-2 and half are applied using electrodes 3-4, the result of the multiplexing results in 30 Hz delivered through electrodes 1-2 and 30 Hz delivered through electrodes 3-4. The target area of vertebrates 3-5 still receive an effective therapy at 60 Hz using electrodes 1-4 with the lead 408 positioned as desired. Although more precisely, vertebrates 3-4 may receive pulses at 30 Hz and vertebrates 4-5 may receive pulses at 30 Hz. If the lead shifts down as indicated by the arrow, electrodes 3-4 may be out of range, but the target area of vertebrates 3-5 will still be receiving pulses at 30 Hz through electrode 1-2. While the efficacy is not the desired efficacy after the shift of the lead, the patient is still receiving therapy that is 75-90% as effective as the desired efficacy. A modification of the approach is to multiplex the therapy pulses using electrodes 1 and 3 as the first pair and electrodes 2 and 4 as the second pair to broaden the therapy area treated by one electrode pair. Although the area provided the therapy by one pair is broadened in the event of a lead shift, the change in electrode spacing may reduce the efficacy of the delivered pulses.

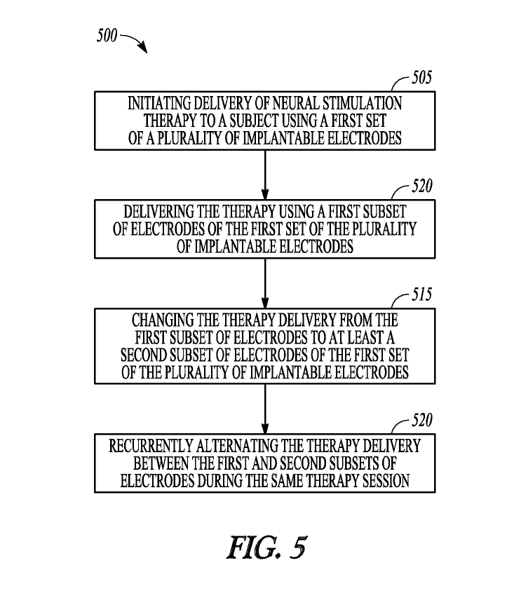

FIG. 5 shows a flow diagram of an example of a method 500 of maintaining efficacy of NST, such as when there is a shift in lead position. At block 505, delivery of electrical autonomic neural modulation therapy to a subject is initiated using an IMD. The therapy is delivered to a first set of a plurality of implantable electrodes during a therapy session. In the example of FIG. 4, electrodes 1-4 can be viewed as the first set of the plurality of implantable electrodes.

At block 510, NST is delivered using a first subset of electrodes of the first set of the plurality of implantable electrodes. In the Example of FIG. 4, the pair of electrodes 1 and 2 can be viewed as the first subset of the set of electrodes 1-4. Either electrode 1 or 2 can be used as the anode or as the cathode of the electrode pair. In certain examples, a subset can include more than two electrodes, but two electrodes are described to simplify the description.

At block 515, the delivery of NST is changed from the first subset of electrodes to at least a second subset of electrodes of the first set of the plurality of implantable electrodes. The pair of electrodes 3 and 4 can be viewed as the second subset of electrodes. At least one electrode of the second subset of electrodes is different from the first subset of electrodes. Thus, electrodes 2 and 3 could be the second subset of electrodes, or electrodes 1 and 4 could be the second subset of electrodes and so on. In some examples, where one electrode is common (e.g., electrodes 1 and 2 in FIG. 4 are the first subset and electrodes 2 and 3 are the second subset), the common electrode (e.g., electrode 2) can be used as both an anode for the first subset and a cathode for the second subset.

At block 520, the therapy delivery is recurrently alternated between the first and second subsets of electrodes during the same therapy session. In some examples, the therapy delivery is evenly divided between the two subsets of electrodes. Thus as in the example above, a 60 Hz therapy source could be divided into two 30 Hz deliveries of therapy using two subsets of electrodes. Other permutations are within contemplation of the method 500. In an illustrative example, the switching between the two subsets is not even, such as delivering NST two thirds of the time using the first subset and one third of the time using the second subset. A 60 Hz therapy source could be divided into one 40 Hz delivery and one 20 Hz delivery when multiplexed in this way.

Note that the combined stimulation of the last example provides 60 Hz therapy that oscillates among vertebrates numbered 3-6. Because four electrodes of the lead 208 are used instead of two, a shift in the lead in either the superior or inferior direction may still provide therapy that is effective to the subject.

Nerves can adapt to the stimulation and effectiveness of neural stimulation therapy may diminish over time. The neural stimulation can be varied to prevent adaptation of the nerves to the artificial stimulation. For example, the amplitude, frequency, wave morphology, burst frequency and/or duration can be adjusted to abate adaptation. Because the therapy oscillates among vertebrates numbered 3-6, adaptation of the neural tissue target may be reduced.

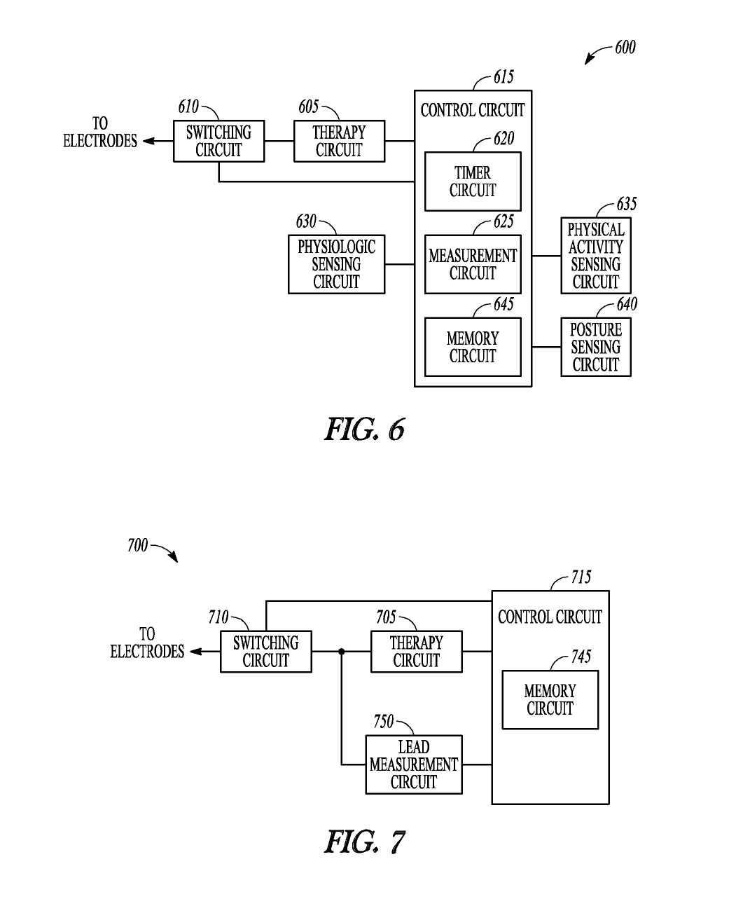

FIG. 6 shows a block diagram of portions of an example of a medical device 600 that maintains efficacy of NST. The device 600 includes a therapy circuit 605 and a switching circuit 610. The switching circuit 610 is connectable to implantable electrodes and the switching circuit is communicatively coupled to the therapy circuit 605. The communicative coupling allows the switching circuit 610 to receive electrical signals from the therapy circuit 605 even though there may be intervening circuitry between the therapy circuit 605 and switching circuit 610. The therapy circuit 605 provides NST to the subject using a first set of a plurality of implantable electrodes. The switching circuit 610 changes the delivery of therapy among the plurality of implantable electrodes. In some examples, the switching circuit 610 includes a multiplexing circuit.

The device 600 also includes a control circuit 615 communicatively coupled to the therapy circuit 605 and switching circuit 610. The control circuit 615 can include a processor such as a microprocessor, a digital signal processor, application specific integrated circuit (ASIC), or other type of processor, interpreting or executing instructions in software modules or firmware modules. The control circuit 615 includes other circuits or sub-circuits to perform the functions described. These circuits may include software, hardware, firmware or any combination thereof. Multiple functions can be performed in one or more of the circuits or sub-circuits as desired.

The control circuit 615 is configured (e.g., programmed) to initiate delivery of the neural stimulation therapy to a first subset of the first set of electrodes during a therapy session. The control circuit 615 then changes the therapy delivery from the first subset of electrodes to at least a second subset of electrodes of the first set of the plurality of implantable electrodes, and recurrently alternates the therapy delivery between the first and second subsets of electrodes during the same therapy session. An example of the operation of the control circuit 615 was described previously in regard to FIG. 4 where electrodes 1 and 2 are included in the first subset and electrodes 3 and 4 are included in the second subset.

In some examples, the control circuit 615 includes a timer circuit 620. The timer circuit 620 can be integral to or communicatively coupled to the control circuit 615. The control circuit 615 initiates delivery of the AMT using the first and second subsets of the first set of the plurality of implantable electrodes during the therapy session. In the example of FIG. 2, electrodes 1-4 can be the first set of electrodes, electrodes 1-2 can be the first subset of electrodes, and electrodes 3-4 can be the second subset. The control circuit 615 changes delivery of the therapy to first and second subsets of a second set of the plurality of implantable electrodes after a specified time duration. In the example of FIG. 2, electrodes 5-8 can be the second set of electrodes, and electrodes 5-6 can be the first subset of electrodes and electrodes 7-8 can be the second subset. At least one electrode of the second set of electrodes is different from the first set of electrodes. Thus, the second set of electrodes may be electrodes 3-6 in FIG. 2, with electrodes 3 and 4 as the first subset and electrodes 5-6 as the second subset.

In some examples, more than two subsets could be used. In some examples, the control circuit 615 multiplexes the therapy delivery among a first subset of electrodes, a second subset of electrodes, and a third subset of electrodes of the first set of the plurality of implantable electrodes. The third subset of electrodes includes at least one electrode different from the first and second subsets of electrodes.

Returning to the example of FIG. 2, the set of implantable electrodes can include electrodes 1-8 of multi-electrode lead 208. The first subset of electrodes can be electrodes 3 and 4, the second subset of electrodes can be electrodes 2 and 3, and the third subset of electrodes can be electrodes 4 and 5. Thus, the first subset of electrodes is arranged between the second and third subsets of electrodes. The AMT may center on the first subset and oscillate to the second and third subsets which are arranged to either side of the first or center subset.

Common electrodes 3 and 4 may serve as both anode and cathode. For instance, electrode 3 may serve as the anode for subset one and serve as the cathode for subset two. Each of three subsets delivers stimulus pulses at 20 Hz, but the combined effect is to provide therapy at 60 Hz. In certain examples, AMT is provided to a specified primary tissue area of the subject using the first subset of electrodes and the autonomic neural modulation therapy is delivered to specified secondary tissue areas using the second and third subsets of electrodes.

In some examples, the electrodes are configured to provide multipolar stimulation. For instance, electrodes 5, 6, and 7 may form one subset of electrodes with electrodes 5 and 7 serving as the cathode and electrode 6 serving as the anode. If it becomes necessary to change the subset, electrodes 6, 7, and 8 may be the electrodes of the subset with electrodes 6 and 8 serving as the cathode and electrode 7 serving as the anode. In some examples, the electrodes are configured to provide unipolar stimulation, such as by using an electrode formed on the housing of the device 600 serving as the anode. The unipolar configuration can be changed by the device 600 by deactivating the can electrode as the activating one or more other electrodes for the anode.

Feedback can be used to determine when electrodes should be changed from the first set of electrodes to the second set of electrodes. According to some examples, the control circuit 615 includes a measurement circuit 625 that measures at least one physiologic parameter of the subject. The measurement circuit 625 can be communicatively coupled to a physiologic sensing circuit 630 which provides an electrical signal representative of the physiologic parameter to be measured.

In certain examples, the physiologic sensing circuit 630 can include a cardiac signal sensing circuit. The cardiac signal sensing circuit provides an electrical cardiac signal representative of electrical activity of the heart of the subject. The device 600 can be coupled to one or more cardiac leads to couple the device to the heart. The cardiac leads can include a proximal end that is coupled to the device and a distal end, coupled by electrodes to one or more portions of a heart. The electrodes can be configured to deliver cardioversion, defibrillation, pacing, or resynchronization therapy, or combinations thereof to at least one chamber of the heart. The electrodes can be electrically coupled to sense amplifiers to sense electrical cardiac signals.

Sensed electrical cardiac signals can be sampled to create an electrogram (sometimes called an egram). An electrogram can be analyzed by the device and/or can be stored in the device and later communicated to an external device where the sampled signals can be analyzed and/or displayed. Using the sensed electrical cardiac signal, the measurement circuit 625 can measure cardiac activity and provide a measure of cardiac activity as the measured physiologic parameter. Examples of the measure of cardiac activity include, among other things, depolarization rate, variability in the depolarization rate, depolarization rate turbulence, a measure of the interval from the P-wave to the R-wave in a sensed cardiac signal, velocity of a T-wave in the sensed cardiac signal, and duration of a depolarization.

In certain examples, the physiologic sensing circuit 630 can include a blood pressure sensing circuit that provides an electrical signal representative of blood pressure of the subject, and the measurement circuit 625 provides a measure of blood pressure of the subject as the physiologic parameter. In certain examples, the physiologic sensing circuit 630 can include a respiration sensing circuit configured to provide an electrical signal representative of subject respiration. The measurement circuit 625 provides a measure of at least one of minute volume or tidal volume of the subject as the physiologic parameter.

In certain examples, the physiologic sensing circuit 630 can include a heart sound sensing circuit that provides an electrical signal representative of one or more heart sounds. Examples of a heart sound sensing circuit include an accelerometer, a strain gauge, and a microphone. Heart sounds are associated with mechanical activity of a patient's heart. The first heart sound (S1) is the sound made by the heart during the near simultaneous closure of the mitral and tricuspid valves. The second heart sound (S2) marks the beginning of diastole. The third heart sound (S3) and fourth heart sound (S4) are related to filling pressures of the left ventricle during diastole. The measurement circuit 625 provides a measure of a parameter of one or more heart sounds as the physiologic parameter.

In certain examples, the physiologic sensing circuit 630 can include a vibration sensor (e.g., an accelerometer) configured for placement to monitor laryngeal vibration. A change in laryngeal vibration can be used to detect vagus nerve stimulation. The measurement circuit 625 provides a measure of laryngeal vibration as the physiologic parameter.

The measured physiologic parameter or parameters can be used to monitor efficacy of the NST and provide feedback for the device 600. The control circuit 615 initiates a plurality of neural stimulation therapy sessions to the subject. For example, the stimulation of an autonomic neural target such as the vagus nerve or a branch thereof may affect heart rate, blood pressure and respiration. A comparator circuit can be used to compare the sensed physiological response to a target range stored in the memory or to a previously measured baseline value. The function of the comparator can be performed within the control circuit 615. The target range stored in the memory can be programmable.

The control circuit 615 receives a comparison result and controls the neural stimulation based on the comparison in an attempt to keep the response within the target range or within a specified range of the baseline. The control circuit 615 changes the therapy delivery from the subsets of the first set of electrodes to first and second subsets of a second set of the plurality of implantable electrodes according to any detected change in the measure of the at least physiologic parameter or when the measure falls outside of the target range.

At least one electrode of the second set of electrodes can be different from the first set of electrodes. Thus, in a non-limiting example, the first set of electrodes can include electrodes 1-4 in FIG. 2 and the second set of electrodes can include electrodes 3-6. The control circuit can initiate NST by switching between electrodes 1-2 as the one subset and electrodes 3-4 as the other subset. If the measured parameter changes by a specified threshold this may indicate that the efficacy of the NST has decreased. In response to the detected change, the control circuit 615 changes the therapy to switch among electrodes of the second set of electrodes and uses electrodes 3-4 as one subset and electrodes 5-6 as the other subset. In another example, electrodes 1-4 can be used as the first set of electrodes and electrodes 5-8 can be used as the second set of electrodes. In another example, electrodes 1-4 can be used as first set of electrodes and electrodes 2-5 can be used as the second set of electrodes.

According to some examples, a measure of physical activity of the subject is used together with the measure of the physiologic parameter to determine whether the delivery of NST needs to be adjusted by changing therapy electrodes. The device 600 can include a physical activity sensing circuit 635 that provides an indication representative of activity of the subject. An example of a physical activity sensing circuit 635 includes an accelerometer or a vibration sensor. The control circuit 615 changes the therapy delivery from switching among subsets of the first set of electrodes to switching among first and second subsets of a second set of the plurality of implantable electrodes. The change is made according to the measured change in the at least one physiologic parameter and a determined physical activity level of the subject. Using a combination of physical activity and efficacy as measured by the physiologic parameter may provide a better indication that a change in the therapy delivery is needed.

According to some examples, a determination of posture of the subject is used together with the measure of the physiologic parameter to determine whether the delivery of NST needs to be adjusted. The device 600 can include a posture sensing circuit 640 that provides an indication according to posture of the subject. An example of a posture sensing circuit 640 includes an accelerometer or a tilt switch. The control circuit 615 changes the therapy delivery from switching among subsets of the first set of electrodes to first and second subsets of a second set of the plurality of implantable electrodes according to the measured change in the at least one physiologic parameter and a determined posture of the subject.

The electrode combinations can be indexed according to posture. In some examples, the device 600 includes a memory circuit 645 integral to or communicatively coupled to the control circuit 615. The memory circuit 645 can store an indication of a set of the plurality of implantable electrodes in association with an indication of a posture of the subject. The control circuit 615 selects the second set of the plurality of implantable electrodes according to the stored indication of the posture of the subject. In the example of FIG. 2, the memory circuit 645 may indicate that a first set of electrodes comprising electrodes 1-4 be used to deliver the NST when the subject is determined to be standing, and indicate that a second set of electrodes comprising electrodes 3-6 be used when the subject is determined to be sitting.

According to some examples, NST may be provided using a dedicated pair of electrodes that are positioned for optimized delivery of NST. The switching or multiplexing of the NST among different subsets of electrodes may be initiated when a shift in lead position is detected. FIG. 7 shows a block diagram of portions of another example of a medical device 700 that maintains efficacy of NST. As in the device 600 of FIG. 6, the device 700 of FIG. 7 includes a therapy circuit 705 to deliver NST, a switching circuit 710, and a control circuit 715. The switching circuit 810 is connectable to plurality of implantable electrodes that are included in a multi-electrode lead, such as the multi-electrode leads of FIGS. 2-4.

The control circuit 715 includes a memory circuit 745 communicatively coupled to, or integral to, the control circuit 715 and stores estimated electrical parameter data associated with electrodes of the multi-electrode lead. The device 700 also includes a lead measurement circuit 750 communicatively coupled to the control circuit 715 and the multi-electrode lead. The lead measurement circuit 750 measures electrical parameter data in response to electrical energy transmitted using the multi-electrode lead. In certain examples, the electrical parameter data can include one or more resistance measurements and capacitance measurements. The electrical parameter or parameter can be measured relative to another device, such as a second implantable lead for example. A shift in the position of the lead relative to the second device may cause a change from the stored values of the electrical parameters.

The control circuit 715 detects a change in position of an implantable lead using a comparison of the measured electrical parameter data and the stored estimated electrical parameter data. When a change is detected, the control circuit 715 changes delivery of the NST from the first and second subsets of the first set of the plurality of implantable electrodes to first and second subsets of a second set of the plurality of implantable electrodes according to the detected change in position of the multi-electrode lead.

According to some examples, one or more of the devices 600, 700 of FIGS. 6-7 may provide NST through a dedicated pair of electrodes that are positioned for optimized delivery of NST. The devices may include a physical activity sensor communicatively coupled to a controller circuit. The control circuit of the devices may change delivery of the NST to switching the stimulation pulse among different subsets of electrodes when detecting that the subject is active, such as by detecting a physical activity level that exceeds a specified threshold activity level. In certain examples, three subsets of electrodes are used and the original dedicated pair may be the center subset. When activity is detected, the NST oscillates among the three subsets with the dedicated pair as the center subset. The control circuit may return the NST delivery back to the dedicated pair of electrodes when the control circuit detects that the subject is more stationary, such as when the physical activity level drops below the specified threshold activity level.

A continual switching, sweeping, or oscillation among different sets of electrodes can maintain delivery of an effective level of NST despite undesired movement of implanted electrodes. Additionally, altering of the electrodes used to deliver NST over sustained periods may reduce adaptation of the nerve tissue receiving the NST. This results in a greater confidence in the efficacy of device-based neural stimulation therapy.

### Additional Notes and Examples

Example 1 includes subject matter (such as an apparatus) comprising a therapy circuit, a switching circuit, and a control circuit. The therapy circuit is configured to provide electrical neural stimulation therapy to a subject using a first set of a plurality of implantable electrodes, and the switching circuit is communicatively coupled to the therapy circuit and configured to change the delivery of therapy among the plurality of implantable electrodes. The control circuit can be configured to initiate delivery of the neural stimulation therapy to a first subset of the first set of electrodes during a therapy session, change the therapy delivery from the first subset of electrodes to at least a second subset of electrodes of the first set of the plurality of implantable electrodes, and recurrently alternate the therapy delivery between the first and second subsets of electrodes during the same therapy session.

In Example 2, the subject matter of Example 1 can optionally include a control circuit having a timer circuit. The control circuit can optionally be configured to initiate delivery of the neural stimulation therapy using the first and second subsets of the first set of the plurality of implantable electrodes during the therapy session, and change delivery of the therapy to first and second subsets of a second set of the plurality of implantable electrodes after a specified time duration during the same therapy session, wherein at least one electrode of the second set of electrodes is different from the first set of electrodes.

In Example 3, the subject matter of one or any combination of Examples 1 and 2 can optionally include a control circuit having a measurement circuit configured to measure at least one physiologic parameter of the subject. The control circuit can optionally be configured to initiate a plurality of neural stimulation therapy sessions, detect a change in the measure of the at least one physiologic parameter of the subject during the plurality of therapy sessions, and change the therapy delivery to first and second subsets of a second set of the plurality of implantable electrodes according to the detected change in the measure of the at least one physiologic parameter, wherein at least one electrode of the second set of electrodes is different from the first set of electrodes.

In Example 4, the subject matter of one or any combination of Examples 1-3 can optionally include a physical activity sensing circuit configured to provide an indication representative of activity of the subject. The control circuit can optionally be configured to change the therapy delivery to first and second subsets of a second set of the plurality of implantable electrodes according to the measured change in the at least one physiologic parameter and a determined physical activity level of the subject.

In Example 5, the subject matter of one or any combination of Examples 1-4 can optionally include a posture sensing circuit configured to provide an indication according to posture of the subject. The control circuit can optionally be configured to change the therapy delivery to first and second subsets of a second set of the plurality of implantable electrodes according to the measured change in the at least one physiologic parameter and a determined posture of the subject.

In Example 6, the subject matter of one or any combination of Examples 1-5 can optionally include a memory circuit integral to or communicatively coupled to the control circuit. The memory circuit can optionally be configured to store an indication of a set of the plurality of implantable electrodes in association with an indication of a posture of the subject, and the control circuit can optionally be configured to select the second set of the plurality of implantable electrodes according to the stored indication of the posture of the subject.

In Example 7, the subject matter of one or any combination of Examples 1-6 can optionally include at least one of a cardiac signal sensing circuit, a blood pressure sensing circuit, a respiration sensing circuit, a vibration sensing circuit, and a heart sound sensing circuit. The cardiac signal sensing circuit is configured to provide an electrical signal representative of electrical activity of the heart of the subject, and the measurement circuit can be configured to measure at least one of a depolarization rate of the subject or variability in the depolarization rate of the subject. The blood pressure sensing circuit is configured to provide an electrical signal representative of blood pressure of the subject, and the measurement circuit can be configured to provide a measure of blood pressure of the subject. The respiration circuit is configured to provide an electrical signal representative of subject respiration, and the measurement circuit can be configured to provide a measure of at least one of minute volume or tidal volume of the subject. The heart sound sensing circuit is configured to provide an electrical signal representative of one or more heart sounds, and the measurement circuit can be configured to provide a measure of a parameter of one or more heart sounds.

In Example 8, the subject matter of one or any combination of Examples 1-7 can optionally include the plurality of implantable electrodes being included in a multi-electrode lead. The subject matter can further include a memory circuit and a lead measurement circuit. The memory circuit can be communicatively coupled to, or integral to, the control circuit and configured to store estimated electrical parameter data associated with electrodes of the multi-electrode lead. The lead measurement circuit can be communicatively coupled to the control circuit and the multi-electrode lead, and configured to measure electrical parameter data in response to electrical energy transmitted using the multi-electrode lead. The control circuit can optionally be configured to detect a change in position of a multi-electrode lead using a comparison of the measured electrical parameter data and the stored estimated electrical parameter data, and change delivery of the neural stimulation therapy from the first and second subsets of the first set of the plurality of implantable electrodes to first and second subsets of a second set of the plurality of implantable electrodes according to the detected change in position of the multi-electrode lead.

In Example 9, the subject matter of one or any combination of Examples 1-8 can optionally include a control circuit configured to multiplex the therapy delivery among the first subset of electrodes, the second subset of electrodes, and a third subset of electrodes of the first set of the plurality of implantable electrodes. The third subset of electrodes includes at least one electrode different from the first and second subsets of electrodes, and the first subset of electrodes is arranged between the second and third subsets of electrodes.

In Example 10, the subject matter of one or any combination of Examples 1-9 can optionally include the plurality of implantable electrodes configured as a multi-electrode lead to provide the neural stimulation therapy.

Example 11 can include subject matter (such as a method, a means for performing acts, or a machine-readable medium including instructions that, when performed by the machine, cause the machine to perform acts), or can optionally be combined with the subject matter of one or any combination of Examples 1-10 to include such subject matter comprising initiating delivery of electrical autonomic neural modulation therapy to a subject using an IMD (wherein the therapy is delivered to a first set of a plurality of implantable electrodes during a therapy session), delivering the therapy using a first subset of electrodes of the first set of the plurality of implantable electrodes, changing the therapy delivery from the first subset of electrodes to at least a second subset of electrodes of the first set of the plurality of implantable electrodes (wherein at least one electrode of the second subset of electrodes is different from the first subset of electrodes), and recurrently alternating the therapy delivery between the first and second subsets of electrodes during the same therapy session.

Such subject matter can include means for initiating delivery of electrical neural stimulation therapy to a subject, an illustrative example of which includes a control circuit and a therapy circuit. Such subject matter can include means for delivering the therapy using a first subset of electrodes of the first set of the plurality of implantable electrodes, an illustrative example of which includes a therapy circuit that can be electrically coupled to electrodes configured by shape and size for delivering the therapy. Such subject matter can include means for changing the therapy delivery from the first subset of electrodes to at least a second subset of electrodes of the first set of the plurality of implantable electrodes, illustrative examples of which include a switching circuit and a multiplexing circuit. Such subject matter can include means for recurrently alternating the therapy delivery between the first and second subsets of electrodes during the same therapy session, illustrative examples of which can include a switching circuit and a multiplexing circuit electrically coupled to a control circuit.

In Example 12, the subject matter of Example 11 can optionally include delivering the neural stimulation therapy using the first and second subsets of the first set of the plurality of implantable electrodes for at least a first therapy session, and changing delivery of the therapy, after a specified time duration, to first and second subsets of a second set of the plurality of implantable electrodes, wherein at least one electrode of the second set of electrodes is different from the first set of electrodes.

In Example 13, the subject matter of one or any combination of Examples 11 and 12 can optionally include providing a plurality of neural stimulation therapy sessions to the subject, measuring a change in at least one physiologic parameter of the subject during the plurality of therapy sessions, and changing the therapy delivery to first and second subsets of a second set of the plurality of implantable electrodes according to the measured change in the at least one physiologic parameter, wherein at least one electrode of the second set of electrodes is different from the first set of electrodes.

In Example 14, the subject matter of one or any combination of Examples 11-13 can optionally include monitoring a physical activity level of the subject using the IMD, and changing the therapy delivery to the first and second subsets of the second set of the plurality of implantable electrodes according to the measured change in the at least physiologic parameter and the physical activity level of the subject.

In Example 15, the subject matter of one or any combination of Examples 11-14 can optionally include determining posture of the subject using the IMD, and changing the therapy delivery to the first and second subsets of the second set of the plurality of implantable electrodes according to the measured change in the at least physiologic parameter and the determined posture of the subject.

In Example 16, the subject matter one or any combination of Examples 11-15 can optionally include storing an indication of a posture of the subject in association with an indication of a set of the plurality of implantable electrodes, and selecting the second set of the plurality of implantable electrodes according to the stored indication of the posture of the subject.

In Example 17, the at least one physiologic parameter of one or any combination of Examples 13-16 can optionally include at least one of a cardiac depolarization rate of the subject, variability of depolarization rate of the subject, blood pressure of the subject, minute volume of the subject, tidal volume of the subject, and a parameter associated with a heart sound.

In Example 18, the subject matter of one or any combination of Examples 11-17 can optionally include detecting a change in position of a multi-electrode lead, wherein the plurality of implantable electrodes are included in the multi-electrode lead, and changing delivery of the neural stimulation therapy from the first and second subsets of the first set of the plurality of implantable electrodes to first and second subsets of a second set of the plurality of implantable electrodes according to the change in position of the multi-electrode lead.

In Example 19, the subject matter of one or any combination of Examples 11-18 can optionally include multiplexing the therapy delivery among the first subset of electrodes, the second subset of electrodes, and a third subset of electrodes of the first set of the plurality of implantable electrodes. The third subset of electrodes includes at least one electrode different from the first and second subsets of electrodes, and the first subset of electrodes can optionally be arranged between the second and third subsets of electrodes. In Example 20, the subject matter of Example 19 can include delivering the neural stimulation therapy to a specified primary tissue area of the subject using the first subset of electrodes and delivering the neural stimulation therapy to specified secondary tissue areas using the second and third subsets of electrodes.

Example 21 can include, or can optionally be combined with any portion or combination of any portions of any one or more of Examples 1-20 to include, subject matter that can include means for performing any one or more of the functions of Examples 1-20, or a machine-readable medium including instructions that, when performed by a machine, cause the machine to perform any one or more of the functions of Examples 1-20.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” In the event of inconsistent usages between this document and any documents incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a therapy circuit configured to provide electrical neural stimulation therapy to a neural stimulation target of a subject using a first set of a plurality of implantable electrodes, wherein the first set of the plurality of implantable electrodes provides the neural stimulation to a first region of the neural stimulation target;
a switching circuit communicatively coupled to the therapy circuit and configured to change the delivery of therapy among the plurality of implantable electrodes; and
a control circuit configured to:
initiate delivery of the neural stimulation therapy to a first subset of the first set of electrodes during a therapy session;
change the therapy delivery from the first subset of electrodes to at least a second subset of electrodes of the first set of the plurality of implantable electrodes; and
recurrently alternate the therapy delivery among the first subset and the at least second subsets of electrodes during the same therapy session.

2. The apparatus of claim 1, wherein the control circuit includes a timer circuit and is configured to:
initiate delivery of the neural stimulation therapy using the first and second subsets of the first set of the plurality of implantable electrodes during the therapy session; and
change delivery of the therapy to first and second subsets of a second set of the plurality of implantable electrodes after a specified time duration during the same therapy session, wherein the second set of the plurality of implantable electrodes provides the neural stimulation to a second region of the neural stimulation target and wherein at least one electrode of the second set of electrodes is different from the first set of electrodes.

3. The apparatus of claim 1,
wherein the control circuit includes a measurement circuit configured to measure at least one physiologic parameter of the subject, and
wherein the control circuit is configured to:
initiate a plurality of neural stimulation therapy sessions;
detect a change in the measure of the at least one physiologic parameter of the subject during the plurality of therapy sessions; and
change the therapy delivery to first and second subsets of a second set of the plurality of implantable electrodes according to the detected change in the measure of the at least one physiologic parameter, wherein at least one electrode of the second set of electrodes is different from the first set of electrodes.

4. The apparatus of claim 3, including:
a physical activity sensing circuit configured to provide an indication representative of activity of the subject, and
wherein the control circuit is configured to change the therapy delivery to first and second subsets of a second set of the plurality of implantable electrodes according to the measured change in the at least one physiologic parameter and a determined physical activity level of the subject.

5. The apparatus of claim 3, including:
a posture sensing circuit configured to provide an indication according to posture of the subject, and
wherein the control circuit is configured to change the therapy delivery to first and second subsets of a second set of the plurality of implantable electrodes according to the measured change in the at least one physiologic parameter and a determined posture of the subject.

6. The apparatus of claim 5, including:
a memory circuit integral to or communicatively coupled to the control circuit, wherein the memory circuit is configured to store an indication of a set of the plurality of implantable electrodes in association with an indication of a posture of the subject, and
wherein the control circuit is configured to select the second set of the plurality of implantable electrodes according to the stored indication of the posture of the subject.

7. The apparatus of claim 3, including at least one of:
a cardiac signal sensing circuit configured to provide an electrical signal representative of electrical cardiac activity of the heart of the subject, wherein the measurement circuit is configured to measure at least one parameter related to cardiac activity;
a blood pressure sensing circuit configured to provide an electrical signal representative of blood pressure of the subject, wherein the measurement circuit is configured to provide a measure of blood pressure of the subject;
a respiration sensing circuit configured to provide an electrical signal representative of subject respiration, wherein the measurement circuit is configured to provide a measure of at least one of minute volume or tidal volume of the subject;
a heart sound sensing circuit configured to provide an electrical signal representative of one or more heart sounds, wherein the measurement circuit is configured to provide a measure of a parameter of one or more heart sounds; and
a vibration sensing circuit configured to provide an electrical signal representative of laryngeal vibration, wherein the measurement circuit is configured to provide a measure of laryngeal vibration.

8. The apparatus of claim 1,
wherein the plurality of implantable electrodes are included in a multi-electrode lead,
wherein the apparatus includes:
a memory circuit communicatively coupled to, or integral to, the control circuit and configured to store estimated electrical parameter data associated with electrodes of the multi-electrode lead; and
a lead measurement circuit communicatively coupled to the control circuit and the multi-electrode lead, and configured to measure electrical parameter data in response to electrical energy transmitted using the multi-electrode lead, and
wherein the control circuit is configured to:
detect a change in position of a multi-electrode lead using a comparison of the measured electrical parameter data and the stored estimated electrical parameter data; and
change delivery of the neural stimulation therapy from the first and second subsets of the first set of the plurality of implantable electrodes to first and second subsets of a second set of the plurality of implantable electrodes according to the detected change in position of the multi-electrode lead.

9. The apparatus of claim 1,
wherein the control circuit is configured to multiplex the therapy delivery among the first subset of electrodes, the second subset of electrodes, and a third subset of electrodes of the first set of the plurality of implantable electrodes,
wherein the third subset of electrodes includes at least one electrode different from the first and second subsets of electrodes, and
wherein the first subset of electrodes is arranged between the second and third subsets of electrodes.

10. The apparatus of claim 1, including the plurality of implantable electrodes configured as a multi-electrode lead to provide the neural stimulation therapy.

11. A method comprising:
initiating delivery of electrical neural stimulation therapy to a neural stimulation target of a subject using an IMD, wherein the therapy is delivered to a first set of a plurality of implantable electrodes during a therapy session and the first set of electrodes provides the neural stimulation to a first region of the neural stimulation target;
delivering the therapy using a first subset of electrodes of the first set of the plurality of implantable electrodes;
changing the therapy delivery from the first subset of electrodes to at least a second subset of electrodes of the first set of the plurality of implantable electrodes, wherein at least one electrode of the second subset of electrodes is different from the first subset of electrodes; and
recurrently alternating the therapy delivery among the first subset and the at least second subset of electrodes during the same therapy session.

12. The method of claim 11,
delivering the neural stimulation therapy using the first and second subsets of the first set of the plurality of implantable electrodes for at least a first therapy session; and
changing delivery of the therapy, after a specified time duration, to first and second subsets of a second set of the plurality of implantable electrodes, wherein the second set of electrodes provides the neural stimulation to a second region of the neural stimulation target and at least one electrode of the second set of electrodes is different from the first set of electrodes.

13. The method of claim 11, including:
providing a plurality of neural stimulation therapy sessions to the subject;
measuring a change in at least one physiologic parameter of the subject during the plurality of therapy sessions; and
changing the therapy delivery to first and second subsets of a second set of the plurality of implantable electrodes according to the measured change in the at least one physiologic parameter, wherein at least one electrode of the second set of electrodes is different from the first set of electrodes.

14. The method of claim 13, including:
monitoring a physical activity level of the subject using the IMD; and
changing the therapy delivery to the first and second subsets of the second set of the plurality of implantable electrodes according to the measured change in the at least physiologic parameter and the physical activity level of the subject.

15. The method of claim 13, including:
determining posture of the subject using the IMD; and
changing the therapy delivery to the first and second subsets of the second set of the plurality of implantable electrodes according to the measured change in the at least physiologic parameter and the determined posture of the subject.

16. The method of claim 15, including:
storing an indication of a posture of the subject in association with an indication of a set of the plurality of implantable electrodes; and
selecting the second set of the plurality of implantable electrodes according to the stored indication of the posture of the subject.

17. The method of claim 13, wherein the at least one physiologic parameter includes at least one of:
a cardiac depolarization rate of the subject;
variability of depolarization rate of the subject;
turbulence of depolarization rate of the subject;
blood pressure of the subject;
minute volume of the subject;
tidal volume of the subject;
laryngeal vibration of the subject; and
a parameter associated with a heart sound.

18. The method of claim 11,
detecting a change in position of a multi-electrode lead, wherein the plurality of implantable electrodes are included in the multi-electrode lead; and
changing delivery of the neural stimulation therapy from the first and second subsets of the first set of the plurality of implantable electrodes to first and second subsets of a second set of the plurality of implantable electrodes according to the change in position of the multi-electrode lead.

19. The method of claim 11,
wherein recurrently alternating the therapy delivery includes multiplexing the therapy delivery among the first subset of electrodes, the second subset of electrodes, and a third subset of electrodes of the first set of the plurality of implantable electrodes,
wherein the third subset of electrodes includes at least one electrode different from the first and second subsets of electrodes, and
wherein the first subset of electrodes is arranged between the second and third subsets of electrodes.

20. The method of claim 19, including delivering the neural stimulation therapy to a specified primary tissue area of the subject using the first subset of electrodes and delivering the neural stimulation therapy to specified secondary tissue areas using the second and third subsets of electrodes.
